(19) United States
(12) Patent Application Publication
(10) Pub. No.: US 2022/0120744 A1
(43) Pub. Date: Apr. 21, 2022

(54) ASSESSING AND TREATING GERM CELL TUMORS AND PARANEOPLASTIC AUTOIMMUNITY

(71) Applicant: Mayo Foundation for Medical Education and Research, Rochester, MN (US)

(72) Inventors: Vanda A. Lennon, Rochester, MN (US); Andrew McKeon, Rochester, MN (US); Thomas J. Kryzer, Mantorville, MN (US); Sean J. Pittock, Rochester, MN (US); Divyanshu Dubey, Rochester, MN (US)

(21) Appl. No.: 17/425,157

(22) PCT Filed: Jul. 24, 2020
(86) PCT No.: PCT/US2020/015040

§ 371 Date: Jul. 22, 2021

Related U.S. Application Data
(60) Provisional application No. 62/796,765, filed on Jan. 25, 2019.

(51) Int. Cl. G01N 33/564; G01N 33/574; G01N 33/68; A61K 35/14; A61P 35/00

(52) U.S. Cl. CPC G01N 33/564; G01N 33/57488; A61P 35/00; A61K 35/14; G01N 33/6896

(57) ABSTRACT

Materials and methods for detecting LUZP4 IgG in a mammal, as well as materials and methods for using LUZP4 as a serological biomarker of germ cell tumors and paraneoplastic autoimmunity, are provided herein. Also provided herein are materials and methods for treating a mammal identified as having anti-LUZP4 autoantibodies.

### CROSS-REFERENCE TO RELATED APPLICATIONS

This application claims benefit of priority from U.S. Provisional Application Ser. No. 62/796,765, filed on Jan. 25, 2019. The disclosure of the prior application is considered part of (and is incorporated by reference in) the disclosure of this application.

### TECHNICAL FIELD

This document relates to materials and methods for using LUZP4 IgG as a serological biomarker of germ cell (e.g., testicular) tumors and paraneoplastic autoimmunity, and materials and methods for assessing and treating seminoma and paraneoplastic autoimmunity associated with LUZP4-specific autoantibodies. For example, this document relates to materials and methods for detecting the presence or absence of LUZP4-specific autoantibodies.

### BACKGROUND

Testicular germ cell tumors (TGCTs) are the most common solid malignancy affecting men between 15 and 44 years of age, and the incidence of these tumors has increased in the last few decades (Ghazarian et al., Andrology 3(1):13-18. 2015; Siegel et al., CA Cancer J Clin. 69(1):7-34, 2019), About 50% of TGCTs are pure seminomas, and the remaining 50% are non-seminomas (Oldenburg et al., Annals Oncol 24(Suppl. 6):vi125-vi132, 2013). Most patients with seminomas respond well to treatment. (Hallemeier et al., Urol Oncol, 32(1):24 el-6, 2014). Mediastinal or retroperitoneal tumor locations can make diagnostic biopsy technically challenging, however, and reliable serological biomarkers for seminomas are lacking. In some cases, TGCT diagnosis can be aided by traditional markers such as alfa-fetoprotein (AFP) and β-human chorionic gonadotropin β-HCG), but these markers are of limited utility because pure seminomas do not make AFP, and fewer than 10% make β-HCG (Gilligan et al., J Clin Oncol, 28(20):3388-3404, 2010). The only paraneoplastic biomarkers that have been associated with TGCT paraneoplastic neurological syndromes are Ma2 IgG (Voltz et al., N Engl J Med, 340(23):1788-1795, 1999) and Klech like protein 11 (KLHL11) IgG (Mandel-Brehm et al., New Engl J Med, 381(1):47-54, 2019). A majority of Ma2 IgG or KLHL11 positive cases have paraneoplastic brainstem and/or limbic encephalitis (Dalmau et al., Brain 127:1831-1844, 2004). Further, only a minority of TGCT patients are Ma-2 or KLHL11 IgG positive. The lack of specific biomarkers affects patient care, particularly for cases with extra-gonadal tumors (Mosharafa et al., J Urol, 169(6):2126-2128, 2003). As a result, patients may be over-treated with unnecessary interventions such as surgery, chemotherapy, and radiation therapy, resulting in increased burden of treatment in terms of morbidity and cost.

### SUMMARY

This document is based, at least in part, on the discovery that LUZP4 IgG is an autoantibody biomarker of TGCT and TGCT-associated paraneoplastic neurological syndromes, such as paraneoplastic encephalitis. Thus, this document provides materials and methods for using LUZP4 IgG to identify mammals as having, or being likely to have, TGCT and paraneoplastic autoimmunity. This document also provides methods and materials for detecting TGCT and TGCT-associated paraneoplastic neurological syndromes associated with LUZP4-specific autoantibodies, as well as methods and materials for treating seminoma and seminoma-associated paraneoplastic autoimmunity associated with LUZP4-specific autoantibodies. In addition, this document provides materials and methods for identifying mammals as experiencing, or being likely to experience, remission from or recurrence of TGCT and paraneoplastic autoimmunity.

In a first aspect, this document features a method for detecting the presence or absence of a LUZP4-specific autoantibody in a biological sample from a mammal. The method can include (a) contacting the biological sample with a LUZP4 polypeptide to form a LUZP4/LUZP4-specific autoantibody complex if the biological sample contains LUZP4-specific autoantibodies, and (b) detecting the presence or absence of the complex. For example, the method can include detecting the presence of the complex. The presence of the LUZP4-specific autoantibody in the biological sample can be associated with seminoma-associated paraneoplastic neurological syndrome associated with LUZP4-specific autoantibodies in the mammal. The paraneoplastic neurological syndrome can be a paraneoplastic encephalitis. The method can include performing a Western blot to detect the complex. The biological sample can be selected from the group consisting of whole blood, serum, plasma, peripheral blood mononuclear cells (PBMC), and cerebrospinal fluid.

In another aspect, this document features a kit containing a LUZP4 polypeptide and an anti-IgG antibody. The anti-IgG antibody can be an anti-human IgG antibody. The anti-human IgG antibody can include a covalently attached label. The kit can include an anti-LUZP4 antibody.

In another aspect, this document features a method of treating a mammal having a TGCT or TGCT-associated paraneoplastic autoimmunity associated with LUZP4-specific autoantibodies. The method can include (a) withdrawing a biological fluid from the mammal, wherein the biological fluid contains LUZP4-specific autoantibodies, (b) contacting the biological fluid with a LUZP4 polypeptide to remove a substantial portion of the LUZP4-specific autoantibodies from the biological fluid, and (c) returning the biological fluid to the mammal. The mammal can be a human. The biological fluid can be whole blood, serum, plasma, or cerebrospinal fluid. The mammal can have seminoma-associated paraneoplastic autoimmunity (e.g., paraneoplastic encephalitis).

In still another aspect, this document features a method that includes providing a mammal with treatment for seminoma or seminoma-associated paraneoplastic neurological syndrome, where the mammal has been identified as having a biological sample that contains a LUZP4-specific autoantibody. The mammal can be a human. The treatment can include one or more of chemotherapy, radiation, immunosuppressive therapy, and surgery. The biological sample can be selected from the group consisting of whole blood, serum, plasma, PBMC, and cerebrospinal fluid.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a western blot showing the detection of a common antibody among patients with paraneoplastic syndrome and seminoma (lanes 1-9) on a nuclear preparation of TCam2 cell lines. The antibody was not detected in control serum (lane 10). The acid-eluted antibody from the 37-40 KDa band was confirmed by screening again the TCam2 nuclear preparation (lane 11). N, normal.

FIG. 2A is a western blot demonstrating specific binding of patient IgG to LUZP4 overexpression lysate, but no binding of control human serum IgG (N).

FIGS. 2B and 2C are images showing co-localization of immunofluorescence signal for commercial LUZP4 mouse monoclonal IgG (FIG. 2B) and patient IgG (FIG. 2C) in a cell-based assay with LUZP4 overexpression.

FIGS. 3A and 3B are graphs plotting LUZP4 IgG seropositivity in various groups. PNS, paraneoplastic neurological syndrome; NSGCT, non-seminomatous germ cell tumor; SCC, small cell cancer; ANNA1, antineuronal nuclear antibodies type 1 (also referred to as anti-Hu); PCA1, purkinje cell cytoplasmic antibody type 1 (also referred to as anti-Yo); GCT, germ cell tumor; TGCT, testicular germ cell tumors; HGG, hypergamma globulinemia.

FIGS. 4A and 4B are a pair of images showing paraffin sections of human brain stem stained with LUZP4 mouse monoclonal antibody at 10× (FIG. 5A) and 20× (FIG. 5B) magnification.

FIGS. 5A and 5B are a pair of images showing paraffin sections from a seminoma of a patient with paraneoplastic brain-stem encephalitis, stained with LUZP4 mouse monoclonal antibody (FIG. 2A). Seminoma infiltrating lymphocytes were mostly CD3 positive T cells (FIG. 2B).

### DETAILED DESCRIPTION

As described herein, a specific IgG autoantibody to was found in serum and/or cerebrospinal fluid (CSF) of patients presenting with paraneoplastic encephalitis that was associated with TGCT or testicular microlithiasis, a potentially pre-malignant condition (von Eckardstein et al., J Androl, 22(5):818-824, 2001; and Derogee et al., Urology, 57(6):1133-1137, 2001). The target of this IgG autoantibody was identified as LUZP4, a testes cancer antigen with limited expression in normal somatic tissue (Hofmann et al., Proc Natl Acad Sci USA, 105(51):20422-20427, 2008). LUZP4 is a leucine-zipper protein that binds the principal mRNA export receptor (Nxf1) and enhances its RNA binding activity.

This document provides methods and materials for assessing and/or treating a mammal having, or suspected of having, TGCT or a TGCT-associated paraneoplastic neurological syndrome. A mammal having TGCT or a paraneoplastic neurological syndrome associated with LUZP4-specific autoantibodies can have an immune system that is producing LUZP4-specific autoantibodies. For example, a mammal (e.g., a human) having a TGCT-associated paraneoplastic neurological syndrome (e.g., paraneoplastic encephalitis) associated with LUZP4-specific autoantibodies can have an immune system that is producing LUZP4-specific autoantibodies. In some cases, this document provides LUZP4 polypeptides having at least one antigenic site recognized by a LUZP4-specific autoantibody (e.g., a LUZP4-specific autoantibody produced by the immune system of a mammal having a TGCT or a paraneoplastic autoimmunity). In some cases, one or more LUZP4 polypeptides can be used to detect the presence or absence of LUZP4-specific autoantibodies in a sample (e.g., a blood sample or CSF sample obtained from a human). For example, one or more LUZP4 polypeptides can be used to assess a sample obtained from a mammal having, or suspected of having, a TGCT or paraneoplastic neurological syndrome for the presence or absence of LUZP4-specific autoantibodies. The presence of LUZP4-specific autoantibodies can be used to identify (e.g., diagnose) a mammal as having a seminoma or a paraneoplastic autoimmunity (e.g., paraneoplastic encephalitis) associated with LUZP4-specific autoantibodies.

In some cases, one or more LUZP4 polypeptides provided herein can be used to treat a mammal having, or suspected of having, a TGCT or a paraneoplastic neurological syndrome. For example, one or more LUZP4 polypeptides can be administered to a mammal having, or suspected of having, paraneoplastic autoimmunity such as paraneoplastic encephalitis, in order to treat the mammal.

Any appropriate mammal can be assessed as described herein (e.g., assessed for the presence or absence of LUZP4-specific autoantibodies) and/or treated as described herein (e.g., treated with one or more LUZP4 polypeptides). Examples of mammals that can be assessed as described herein and/or treated as described herein include, without limitation, humans, non-human primates, monkeys, bovine species, pigs, horses, dogs, cats, rats, and mice. For example, a human can be assessed for the presence or absence of LUZP4-specific autoantibodies as described herein. In some cases, a human identified as having a TGCT or paraneoplastic neurological syndrome and as having LUZP4-specific autoantibodies can be treated with one or more LUZP4 polypeptides as described herein. Without being bound by a particular mechanism, administration of a LUZP4 polypeptide can result in tolerization to reduce the severity or likelihood of a CNS attack, or to avoid a CNS attack. It also is noted that in some cases, a human identified as having a TGCT or paraneoplastic neurological syndrome and as having LUZP4-specific autoantibodies can be treated with T cell receptor therapy targeting LUZP4.

Any appropriate sample from a mammal (e.g., a human) can be assessed as described herein (e.g., assessed for the presence or absence of LUZP4-specific autoantibodies). Examples of samples (e.g., biological samples) that can be assessed as described herein include, without limitation, biological fluids (e.g., blood such as whole blood, serum, or plasma, urine, CSF, synovial fluid, or saliva), cells (e.g., PBMCs), and biological tissues (e.g., brain tissue such as tissue obtained from a brain biopsy). For example, serum can be obtained from a mammal and assessed for the presence or absence of LUZP4-specific autoantibodies.

A mammal having, or suspected of having, any appropriate type of TGCT or paraneoplastic neurological syndrome can be assessed (e.g., for the presence or absence of LUZP4-specific autoantibodies) and/or treated (e.g., by administering a LUZP4 polypeptide to the mammal) using the methods and materials described herein. In some cases, for example, a TGCT-associated neurological syndrome can be a paraneoplastic autoimmune disorder such as paraneoplastic encephalitis. A paraneoplastic autoimmune disorder can affect any appropriate part of a mammal's nervous system, including the central, peripheral, and autonomic nervous system. Nervous system involvement can be multifocal or can involve a single system (e.g., cerebellar degeneration).

Any appropriate LUZP4 polypeptide can be used as described herein (e.g., to detect the presence or absence of LUZP4-specific autoantibodies and/or to treat a mammal having, or suspected of having, TGCT or paraneoplastic autoimmunity). In some cases, a full length LUZP4 polypeptide can be used to assess a sample for the presence or absence of LUZP4-specific autoantibodies. Examples of LUZP4 polypeptide sequences (and the nucleic acids encoding such polypeptides) can be found in the National Center for Biotechnology Information (NCBI) databases (e.g., Gen Pept and GenBank). Examples of human LUZP4 polypeptide sequences include, without limitation, NCBI Accession No. NP_057467 (e.g., Version No. NP_057467.1), which is identified as isoform 1, and NP_001305769 (e.g., Version No. NP_001305769.1), which is identified as isoform 2. These representative LUZP4 sequences are set forth in SEQ ID NO:1 and SEQ ID NO:2, respectively. Isoform 2 lacks an exon in the 5′ coding region as compared to isoform 1, which results in the use of an alternate 5′ most exon and a frameshift in the 5′ coding region as compared to isoform 1. Isoform 2 is shorter and has a different N-terminus than isoform 1.

(SEQ ID NO: 1)
MASFRKLTLSEKVPPNHPSRKKVNFLDMSLDDIIIYKELEGTNA
EEEKNKRQNHSKKESPSRQQSKAHRHRHRRGYSRCRSNSEEGN
HDKKPSQKPSGFKSGQHPLNGQPLIEQEKCSDNYEAQAEKNQG
QSEGNQHQSEGNPDKSEESQGQPEENHHSERSRNHLERSLSQS
DRSQGQLKRHHPQYERSHGQYKRSHGQSERSHGHSERSHGHS
ERSHGHSERSHGHSKRSRSQGDLVDTQSDLIATQRDLIATQKDLI
ATQRDLIATQRDLIVTQRDLVATERDLINQSGRSHGQSERHQRYS
TGKNTITT
(SEQ ID NO: 2)
MLKKKRIKDRTIVKRNRLQDSNQKLIDIAIGEEKCSDNYEAQAE
KNQGQSEGNQHQSEGNPDKSEESQGQPEENHHSERSRNHLERS
LSQSDRSQGQLKRHHPQYERSHGQYKRSHGQSERSHGHSERSH
GHSERSHGHSERSHGHSKRSRSQGDLVDTQSDLIATQRDLIATQ
KDLIATQRDLIATQRDLIVTQRDLVATERDLINQSGRSHGQSERH
QRYSTGKNTITT

In some cases, a fragment of a LUZP4 polypeptide can be used to assess a sample for the presence or absence of LUZP4-specific autoantibodies. For example, a fragment of a LUZP4 polypeptide containing one or more epitopic sites (e.g., B cell epitopes) can be used to assess a sample for the presence or absence of LUZP4-specific autoantibodies. A LUZP4 polypeptide fragment can have any appropriate length. In some cases, for example, a LUZP4 polypeptide fragment have a length from about 25 to about 300 amino acids (e.g., about 25 to about 50 amino acids, about 50 to about 100 amino acids, about 100 to about 150 amino acids, about 150 to about 200 amino acids, about 200 to about 250 amino acids, or about 250 to about 300 amino acids).

This document also provides nucleic acids and constructs encoding LUZP4 polypeptides or fragments of LUZP4 polypeptides described herein. As used herein, “nucleic acid” can refer to RNA, DNA, or a combination thereof. In some cases, a nucleic acid encoding a LUZP4 polypeptide or fragment of a LUZP4 polypeptide described herein can be an isolated nucleic acid. As used herein with respect to nucleic acids, “isolated” refers to (i) a nucleic acid sequence encoding all or part of a LUZP4 polypeptide, but free of coding sequences that normally flank one or both sides of the nucleic acid sequences encoding the LUZP4 in the genome; or (ii) a nucleic acid incorporated into a vector or into the genomic DNA of an organism such that the resulting molecule is not identical to any naturally-occurring vector or genomic DNA. In some cases, a nucleic acid provided herein can encode a fragment of a LUZP4 polypeptide.

Examples of human LUZP4 nucleic acid sequences include, without limitation, NCBI Accession No. NM_016383 (e.g., Version No. NM_016383.5), which is identified as transcript variant 1, and NM_001318840 (e.g., Version No. NM_001318840.1), which is identified as transcript variant 2. Representative LUZP4 nucleic acid sequences are set forth in SEQ ID NO:3 (transcript variant 1) and SEQ ID NO:4 (transcript variant 2).

(SEQ ID NO: 3)
ACACGCGCCCTACCTCGGAGTGTGTGGCGCCATGATGCAGGG
AAGATGGCTTCGTTTCGGAAGCTAACGCTTTCTGAAAAAGTG
CCGCCAAATCATCCCAGTCGGAAAAAGGTTAACTTCCTAGAT
ATGTCTCTAGACGACATTATAATCTATAAAGAGTTAGAAGGGA
CAAATGCTGAAGAAGAAAAGAATAAAAGACAGAACCATAGT
AAAAAGGAATCGCCTTCAAGACAGCAATCAAAAGCTCATAG
ACATCGCCATCGGAGAGGCTACTCAAGATGCAGAAGCAACTC
TGAGGAAGGAAATCATGATAAAAAACCATCCCAAAAACCTTC
TGGATTCAAGTCTGGACAACACCCTTTAAATGGGCAGCCTTT
AATTGAGCAGGAGAAGTGCAGTGACAATTATGAGGCCCAAG
CAGAGAAGAATCAAGGCCAGTCAGAGGGGAACCAGCATCAA
TCAGAAGGAAATCCGGACAAATCAGAAGAATCCCAGGGCCA
ACCAGAAGAAAATCATCATTCTGAGCGATCCCGAAACCACTT
AGAGAGATCTCTTTCTCAGTCAGACAGATCTCAAGGGCAGCT
AAAGAGACATCATCCCCAATATGAGAGATCTCATGGCCAATA
CAAGAGATCTCATGGTCAATCTGAGAGATCTCATGGCCACTC
AGAGAGATCTCATGGTCACTCAGAGAGATCTCATGGTCACTC
AGAGAGATCTCATGGTCACTCAAAGAGATCTCGTAGCCAGGG
AGATCTTGTGGACACTCAGAGTGATCTCATAGCCACTCAGAG
AGATCTCATAGCCACTCAGAAAGATCTCATAGCCACTCAGAG
AGATCTCATAGCCACTCAGAGAGATCTCATAGTCACTCAGAG
AGATCTCGTGGCCACTGAGAGAGATCTCATAAATCAGTCAGG
GAGATCTCATGGCCAATCAGAAAGACATCAGAGATACTCAAC
AGGTAAAAATACAATAACTACTTAATCATCAGAACAATGTGTT
GAATTCTGTGGAAATAGAAAAGCATATATCTATATTCTAATGGC
TAAATATGTATTTGTTGAAACATGTATATTGGGACAAAGACAT
AAATATTAGAATGGAGGTAATACATACATAGTATCAATATTGTT
TCAACTTGATGTCCTCTAAGCTATCATCCAGTTACCCAGGATG
TCCCATTAAGTTGTTCCCGGTAGGTCTGCTTTCCCTGGAAGA
GCCGTATGTACTCAGCCTTTCCTATTGGGCCTTCCCCACAATT
AGAATATTTTGACTTAGTGTCCTGTCCCCCTTGGACGTTCCAA
CTTGACTTAGTGTCCAGTGCCCCTTGGACATTCCAACCTGGT
AGGTAAGCTAATCTAACAACTAACTGCCAAATTGATAATATAT
AATCTATGATAATGAATATCTCTTTTGTGTCTCCTTCCTAAGCC
ATCCTCAGAGAGTCCTTAGCAGACAAATGGTAGATGTATCTTT
GGGCAGCTGAACTTTTCTGCTTTCCTCAAATCAGACCATATGA
GAGGATATATTCTATGCATAGATGTAATGCTAACCTTCTGAATA
TATTTTGAATACATTTATATATTCACTGTTGCCTTATAAAACTGT
TAGGGTAGGTCTGTCTACCCTAGCAAAAGAAACACAGAAATT
TAAATGTACTGGGAGTTATGTTGTTAAAAACACAAGATATGTT
AACTGCAGTTTGTTTGGTTATTCAATAAAAGTTTTAGTTTTAA
TTTCTCA
(SEQ ID NO: 4)
ACGCGCCCTACCTCGGAGTGTGTGGCGCCATGATGCAGGGAA
GATGGCTTCGTTTCGGAAGCTAACGCTTTCTGAAAAAGTGCC
GCCAAATCATCCCAGTCGGAAAAAGGTTAACTTCCTAGATAT
GTCTCTAGACGACATTATAATCTATAAAGAGTTAGAAGGGACA
AATGCTGAAGAAGAAAAGAATAAAAGACAGAACCATAGTAA
AAAGGAATCGCCTTCAAGACAGCAATCAAAAGCTCATAGAC
ATCGCCATCGGAGAGGAGAAGTGCAGTGACAATTATGAGGCC
CAAGCAGAGAAGAATCAAGGCCAGTCAGAGGGGAACCAGC
ATCAATCAGAAGGAAATCCGGACAAATCAGAAGAATCCCAG
GGCCAACCAGAAGAAAATCATCATTCTGAGCGATCCCGAAAC
CACTTAGAGAGATCTCTTTCTCAGTCAGACAGATCTCAAGGG
CAGCTAAAGAGACATCATCCCCAATATGAGAGATCTCATGGC
CAATACAAGAGATCTCATGGTCAATCTGAGAGATCTCATGGC
CACTCAGAGAGATCTCATGGTCACTCAGAGAGATCTCATGGT
CACTCAGAGAGATCTCATGGTCACTCAAAGAGATCTCGTAGC
CAGGGAGATCTTGTGGACACTCAGAGTGATCTCATAGCCACT
CAGAGAGATCTCATAGCCACTCAGAAAGATCTCATAGCCACT
CAGAGAGATCTCATAGCCACTCAGAGAGATCTCATAGTCACT
CAGAGAGATCTCGTGGCCACTGAGAGAGATCTCATAAATCAG
TCAGGGAGATCTCATGGCCAATCAGAAAGACATCAGAGATAC
TCAACAGGTAAAAATACAATAACTACTTAATCATCAGAACAAT
GTGTTGAATTCTGTGGAAATAGAAAAGCATATATCTATATTCTA
ATGGCTAAATATGTATTTGTTGAAACATGTATATTGGGACAAA
GACATAAATATTAGAATGGAGGTAATACATACATAGTATCAATA
TTGTTTCAACTTGATGTCCTCTAAGCTATCATCCAGTTACCCA
GGATGTCCCATTAAGTTGTTCCCGGTAGGTCTGCTTTCCCTGG
AAGAGCCGTATGTACTCAGCCTTTCCTATTGGGCCTTCCCCAC
AATTAGAATATTTTGACTTAGTGTCCTGTCCCCCTTGGACGTT
CCAACTTGACTTAGTGTCCAGTGCCCCTTGGACATTCCAACC
TGGTAGGTAAGCTAATCTAACAACTAACTGCCAAATTGATAAT
ATATAATCTATGATAATGAATATCTCTTTTGTGTCTCCTTCCTAA
GCCATCCTCAGAGAGTCCTTAGCAGACAAATGGTAGATGTAT
CTTTGGGCAGCTGAACTTTTCTGCTTTCCTCAAATCAGACCAT
ATGAGAGGATATATTCTATGCATAGATGTAATGCTAACCTTCTG
AATATATTTTGAATACATTTATATATTCACTGTTGCCTTATAAAA
CTGTTAGGGTAGGTCTGTCTACCCTAGCAAAAGAAACACAGA
AATTTAAATGTACTGGGAGTTATGTTGTTAAAAACACAAGATA
TGTTAACTGCAGTTTGTTTGGTTATTCAATAAAAGTTTTAGTT
TTAAAAAAAAAAAAAAAAAAA

In some cases, a LUZP4 polypeptide that can be used as described herein (e.g., to detect the presence or absence LUZP4-specific autoantibodies and/or to treat a mammal having, or suspected of having, a TGCT or paraneoplastic neurological syndrome) can have a sequence that deviates from a wild type LUZP4 polypeptide sequence, sometimes referred to as a variant sequence. For example, a LUZP4 polypeptide sequence can have at least 80% sequence identity to SEQ ID NO:1 or SEQ ID NO:2, where the polypeptide includes one or more amino acid additions, subtractions, or substitutions compared to SEQ ID NO:1 or SEQ ID NO:2. In some embodiments, a LUZP4 polypeptide sequence can have at least 85% sequence identity, 90% sequence identity, 95% sequence identity, 96% sequence identity, 97% sequence identity, 98% sequence identity, or 99% sequence identity to SEQ ID NO:1 or SEQ ID NO:2, provided that it includes one or more amino acid additions, subtractions, or substitutions compared to SEQ ID NO:1 or SEQ ID NO:2.

Percent sequence identity is calculated by determining the number of matched positions in aligned nucleic acid or polypeptide sequences, dividing the number of matched positions by the total number of aligned nucleotides or amino acids, respectively, and multiplying by 100. A matched position refers to a position in which identical nucleotides or amino acids occur at the same position in aligned sequences. The total number of aligned nucleotides or amino acids refers to the minimum number of LUZP4 nucleotides or amino acids that are necessary to align the second sequence, and does not include alignment (e.g., forced alignment) with non-LUZP4 sequences, such as those fused to LUZP4. The total number of aligned nucleotides or amino acids may correspond to the entire LUZP4 sequence or may correspond to fragments of the full-length LUZP4 sequence as defined herein.

Sequences can be aligned using the algorithm described by Altschul et al. (Nucleic Acids Res., 25:3389-3402 (1997)) as incorporated into BLAST (basic local alignment search tool) programs, available at ncbi.nlm.nih.gov on the World Wide Web. BLAST searches or alignments can be performed to determine percent sequence identity between a LUZP4 nucleic acid molecule and any other sequence or portion thereof using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a LUZP4 sequence and another sequence, the default parameters of the respective programs are used.

A LUZP4 polypeptide (or fragment thereof) that can be used as described herein (e.g., to detect the presence or absence LUZP4-specific autoantibodies and/or to treat a mammal having, or suspected of having, a TGCT or paraneoplastic autoimmunity) can be from any appropriate source. In some cases, a LUZP4 polypeptide (or fragment thereof) described herein can be obtained from human, mouse, or other mammalian neuronal tissue, neuronal cell lines, or transfected cells (e.g., mammalian, E. coli, or yeast cells) expressing a recombinant LUZP4 nucleic acid, or the LUZP4 polypeptide may be synthetic. In some cases, a LUZP4 polypeptide (or fragment thereof) described herein can be in a cell lysate (e.g., a whole cell lysate or a cell fraction). In some cases, a LUZP4 polypeptide (or fragment thereof) described herein can be a purified polypeptide. A “purified” polypeptide refers to a polypeptide that constitutes the major component in a mixture of components, e.g., 30% or more, 40% or more, 50% or more, 60% or more, 70% or more, 80% or more, 90% or more, 95% or more, or 99% or more by weight. Polypeptides may be purified by methods including, without limitation, affinity chromatography or immunosorbent affinity column. Such methods can be modified to increase the solubility of the polypeptide, and purified polypeptides can be examined for their immunogenicity using methods such as western blot or immunoprecipitation assays.

Fragments of a LUZP4 polypeptide can be generated using any appropriate method. For example, given a LUZP4 polypeptide sequence, any appropriate polypeptide fragment can be generated by proteolytic cleavage (e.g., of a full length LUZP4 polypeptide). Fragments of LUZP4 also can be generated by chemical synthesis.

This document also provides methods for detecting LUZP4-specific autoantibodies. The presence of LUZP4-specific autoantibodies can be used to diagnose TGCT or paraneoplastic autoimmune disorders associated with LUZP4-specific autoantibodies. For example, the methods and materials described herein can be used to identify a mammal (e.g., a human) as having a TGCT or paraneoplastic neurological syndrome associated with LUZP4-specific autoantibodies (e.g., having an immune system that is producing LUZP4-specific autoantibodies) based, at least in part, on the presence of LUZP4-specific autoantibodies in a sample obtained from the mammal. In some cases, a LUZP4 polypeptide (or fragment thereof) described herein can be used (e.g., in various immunological techniques) to detect a LUZP4-specific autoantibody. For example, a LUZP4 polypeptide (or fragment thereof) described herein can be used in an immunoassay to detect LUZP4-specific autoantibodies in a biological sample. In some cases, LUZP4 polypeptides (or fragments thereof) described herein can be contacted with a sample (e.g., serum) obtained from a mammal (e.g., a mammal suspected of having a TGCT or paraneoplastic autoimmunity associated with LUZP4-specific autoantibodies) under conditions where, when LUZP4-specific autoantibodies are present, the LUZP4 polypeptides (or fragments thereof) can form a complex with the LUZP4-specific autoantibodies (e.g., a LUZP4/LUZP4-specific autoantibody complex), and an immunoassay can be used to detect the complex. For example, when LUZP4-specific autoantibodies are present in a sample obtained from a mammal suspected of having a TGCT-associated paraneoplastic autoimmunity, a LUZP4 polypeptide (or fragment thereof) described herein can be used to form a complex with the LUZP4-specific autoantibodies (e.g., a LUZP4/LUZP4-specific autoantibody complex), and an immunoassay can be used to detect the complex. Examples of immunoassays that can be used to detect the presence of a LUZP4-specific autoantibody, or a LUZP4/LUZP4-specific autoantibody complex include, without limitation, immunocytochemical staining techniques, immunohistochemical staining techniques, enzyme-linked immunosorbent assays (ELISA), western blotting, radioimmunoassays, cell-based immunofluorescence assays, and flow cytometry.

In addition, this document provides methods for detecting the presence or absence of a LUZP4-specific T-cell response in a biological sample from a mammal. For example, a biological sample (e.g., a blood sample) can be examined to determine whether it contains LUZP4-specific T cells. Any appropriate method can be used to detect LUZP4-specific T cells, including enzyme-linked immunospot (ELISPOT), ELISA, flow cytometry, mass cytometry methods, limiting dilutions culture, intracellular staining, and/or tetramer staining.

In some cases, methods for detecting LUZP4-specific autoantibodies or a LUZP4-specific T-cell response can be used to assess whether a mammal treated for TGCT or a paraneoplastic neurological syndrome is experiencing a recurrence of the condition. For example, detecting the presence of LUZP4 autoantibodies (or the presence of increased levels of LUZP4 autoantibodies) in a mammal treated for TGCT or paraneoplastic neurological syndrome can indicate the recurrence of TGCT or paraneoplastic neurological syndrome. LUZP4 IgG also can be used as a marker for remission from TGCT or paraneoplastic neurological syndrome. For example, a sample can be obtained from a LUZP4 IgG seropositive mammal after the mammal is treated for TGCT or paraneoplastic neurological syndrome (e.g., with chemotherapy, radiation, surgery, or a combination thereof). A reduced level of LUZP4 IgG in the post-treatment sample (e.g., the lack of detectable LUZP4 IgG) can indicate that the mammal is in remission.

Having the ability to identify mammals as having a TGCT or paraneoplastic autoimmunity can allow those mammals to be properly identified and treated in an effective and reliable manner. For example, the treatments provided herein (e.g., LUZP4 polypeptides and chemotherapy, radiation, surgery, or a combination thereof) can be used to treat disease patients identified as having TGCT or paraneoplastic neurological syndrome.

LUZP4 polypeptides (or fragments thereof) can be used as described herein (e.g., to detect the presence or absence of LUZP4-specific autoantibodies and/or to treat a mammal having, or suspected of having, a TGCT or paraneoplastic autoimmunity) with or without modification. In some cases, a LUZP4 polypeptide (or fragment thereof) can be modified for the detection of LUZP4-specific autoantibodies in vitro (e.g., in an immunoassay). In some cases, a LUZP4 polypeptide (or fragment thereof) can be modified for the detection of LUZP4-specific autoantibodies in vivo (e.g., in an in vivo imaging technique). Polypeptides (or fragments thereof) can be labeled by either covalently or non-covalently combining the polypeptide with a second substance that provides for detectable signal. A wide variety of labels and conjugation techniques can be used. Some examples of labels that can be used include radioisotopes, enzymes, substrates, cofactors, inhibitors, fluorescent agents, chemiluminescent agents, magnetic particles, and the like. In some cases, a LUZP4 polypeptide (or fragment thereof) can be conjugated to an imaging agent. Suitable imaging agents include, but are not limited to, radioisotopes, such as ³²P, ⁹⁹Tc, ¹¹¹In, and ¹³¹I.

In some cases, an anti-LUZP4 antibody can be used in various immunological techniques for detecting LUZP4 polypeptides. For example, an anti-LUZP4 antibody can be used in an immunoassay to detect a LUZP4 polypeptide. In some cases, when LUZP4-specific autoantibodies are present in a sample, LUZP4 polypeptides can form a complex with the LUZP4-specific autoantibodies (e.g., a LUZP4/LUZP4-specific autoantibody complex), and an immunoassay using an anti-LUZP4 antibody can be used to detect the complex. Examples of immunoassays that can be used to detect the presence of a LUZP4 polypeptide, or a LUZP4/LUZP4-specific autoantibody complex, include, without limitation, immunocytochemical staining techniques, immunohistochemical staining techniques, ELISA, western blot, radioimmunoassays, cell-based immunofluorescence assays, and flow cytometry.

Anti-LUZP4 antibodies can be used with or without modification for the detection of LUZP4 polypeptides. Anti-LUZP4 antibodies can be labeled either directly or indirectly, and a wide variety of labels, including radioisotopes, enzymes, substrates, cofactors, inhibitors, fluorescent agents, chemiluminescent agents, and magnetic particles. In some cases, an anti-LUZP4 antibody having specific binding affinity for a LUZP4 polypeptide can be conjugated to an imaging agent. Examples of imaging agents that can be used include, without limitation, radioisotopes, such as ³²P, ⁹⁹Tc, ¹¹¹In, and ¹³¹I.

In some cases, a mammal identified as having a TGCT or paraneoplastic neurological syndrome associated with LUZP4-specific autoantibodies (e.g., having an immune system that is producing LUZP4-specific autoantibodies) as described herein (e.g., based, at least in part, on the presence of LUZP4-specific autoantibodies in a sample obtained from the mammal) can have the diagnosis confirmed. For example, identification of a paraneoplastic neurological syndrome can be confirmed using, for example, physical examination such as a neurological examination (e.g., for memory, concentration, vision, hearing, balance, coordination, and/or reflexes), electrophysiological monitoring (e.g., via an electroencephalogram (EEG)), and imaging studies such as brain imaging studies (e.g., computer tomography (CT) scanning and magnetic resonance imagining (MRI)).

This document also provides methods for treating a mammal (e.g., a human, a non-human primate, or a rodent) identified as having a TGCT and/or paraneoplastic neurological syndrome (e.g., whose immune system is producing LUZP4-specific autoantibodies). In some cases, methods for treating a mammal (e.g., a human) identified as having a TGCT or paraneoplastic neurological syndrome associated with the presence of LUZP4-specific autoantibodies can be effective to reduce one or more symptoms of the paraneoplastic neurological syndrome. Examples of symptoms of paraneoplastic neurological syndrome include, without limitation, mood changes, problems sleeping, memory deficits (e.g., severe short-term memory deficits), and seizures, including seizure-like spells or grand mal seizures that result in a total loss of consciousness and neuropathies. In some cases, methods for treating a mammal (e.g., a human) identified as having a TGCT or paraneoplastic neurological syndrome associated with the presence of LUZP4-specific autoantibodies can be effective to remove a substantial portion of LUZP4-specific autoantibodies present within the mammal (e.g., present within a body fluid of the mammal). As used herein, removing a “substantial portion” means removing at least 20% (e.g., at least 20%, at least 30%, at least 40%, at least 50%, at least 60%, at least 65%, at least 70%, at least 75%, at least 80%, at least 85%, at least 90%, at least 93%, at least 95%, at least 96%, at least 97%, at least 98%, at least 99%, at least 99.5%, at least 99.8%, or even 100%) of the LUZP4-specific autoantibodies that were present in the body fluid of a mammal prior to treating the mammal as described herein. The body fluid can be blood (e.g., serum or plasma) or any other body fluid (e.g., lymph or cerebrospinal fluid). In some cases, depleting LUZP4-specific autoantibodies from mammals with a TGCT or paraneoplastic neurological syndrome as described herein also can be effective to reduce one or more symptoms of the TGCT or paraneoplastic neurological syndrome.

In some cases, a mammal identified as having a TGCT or paraneoplastic neurological syndrome can be treated by administering to the mammal any appropriate agent or therapy used to treat TGCT or paraneoplastic neurological syndrome. In some cases, for example, an agent or therapy used to treat TGCT or paraneoplastic neurological syndrome can treat one or more symptoms of the TGCT or paraneoplastic neurological syndrome. Examples of agents and therapies that can be used to treat TGCT or paraneoplastic neurological syndrome include, without limitation, surgical removal of the tumor, radiation therapy, chemotherapy, and combinations thereof. In some cases, LUZP4 antigen specific tolerance induction strategies or immunotherapy targeting LUZP4-specific T cells and/or B cells can be used to treat a paraneoplastic neurological syndrome associated with LUZP4-specific autoantibodies.

In some cases, a mammal identified as having a TGCT or paraneoplastic neurological syndrome associated with the presence of LUZP4-specific autoantibodies can be treated using one or more LUZP4 polypeptides (e.g., in an apheresis method). For example, apheresis for the treatment of a paraneoplastic neurological syndrome associated with LUZP4-specific autoantibodies can be used to remove LUZP4-specific autoantibodies from a human. Methods and extracorporeal systems for apheresis (i.e., the process of withdrawing blood from a mammal, removing components from the blood, and returning the blood, or blood depleted of one or more components, to the mammal) can be used as described elsewhere (see, for example, U.S. Pat. Nos. 4,708,713; 5,258,503; 5,386,734; and 6,409,696). In some cases, an apheresis method can be used to remove LUZP4-specific autoantibodies from a body fluid of a mammal. The method can include withdrawing a body fluid from a mammal, removing a substantial portion of LUZP4-specific autoantibodies from the fluid, and returning the fluid to the mammal. Antibodies removed can be of any class, e.g., IgG (such as IgG1, IgG2, IgG3, IgG4), IgM, IgD, IgA, or IgE antibodies.

Removal of LUZP4-specific autoantibodies can be performed by contacting a body fluid with one or more LUZP4 polypeptides described herein. In some cases, the LUZP4 polypeptide can be bound to a solid support. Such solid supports can be, without limitation, membranes, fibers, spherical beads, or granules and can be made with a water-insoluble, preferably porous, biocompatible material (e.g., one or more organic polymers such as agarose, dextran, and/or polyacrylamide, or one or more inorganic porous materials such as porous glass or porous silica gel). Such materials can be used as is or adapted (e.g., derivatized with appropriate chemical groups) for attachment of a LUZP4 polypeptide.

When the body fluid is blood, the plasma and/or white blood cells can be separated from red blood cells (e.g., erythrocytes), and the red blood cells can be returned to the mammal with or without white blood cells. In some cases, the blood cells are returned to the mammal with artificial rather than their original blood plasma. The “replacement fluid” (e.g., physiological saline) can be administered to the mammal after removal of the fluid. In some cases, the LUZP4-specific autoantibodies can be selectively removed from the blood plasma in the course of apheresis, and the blood cells can be mixed with the LUZP4-specific autoantibody-depleted plasma and then re-infused as a mixture into the mammal.

The system can be a continuous one in which, for example, blood is pumped out of a blood vessel (e.g., an artery or a vein) passed over a solid support derivatized with LUZP4 polypeptides described herein and pumped directly back into a blood vessel of the mammal. As in non-continuous systems, blood cells can be separated from plasma prior to passing of the plasma over the solid support.

In cases where LUZP4 polypeptides described herein are used in an apheresis method to treat a mammal (e.g., a human) having a TGCT or paraneoplastic neurological syndrome, the methods also can include administering to the mammal any appropriate agent or therapy used to treat a TGCT or paraneoplastic neurological syndrome described herein. In cases where a mammal having a TGCT or paraneoplastic neurological syndrome is treated with one or more LUZP4 polypeptides described herein and is treated with agents or therapies used to treat TGCT or paraneoplastic neurological syndrome described herein, the agents or therapies used to treat an TGCT or paraneoplastic neurological syndrome can be administered at the same time or independently. For example, one or more LUZP4 polypeptides described herein and one or more agents or therapies used to treat a TGCT or paraneoplastic neurological syndrome can be formulated together to form a single composition. In some cases, one or more LUZP4 polypeptides described herein can be administered first, and the one or more agents or therapies used to treat TGCT or paraneoplastic neurological syndrome can be administered second, or vice versa.

In some cases, a mammal identified as having a TGCT or paraneoplastic neurological syndrome associated with the presence of LUZP4-specific autoantibodies can be treated with an immunosuppressive therapy. For example, a mammal can be treated to target T-cells or B-cells, in order to prevent progression of LUZP4-associated paraneoplastic autoimmunity. Non-limiting examples of immunosuppressive agents include corticosteroids (e.g., prednisone, budesonide, and prednisolone), kinase inhibitors (e.g., tofacitinib), calcineurin inhibitors (e.g., cyclosporine and tacrolimus), mTOR inhibitors (e.g., sirolimus and everolimus), inosine monophosphate dehydrogenase (IMDH) inhibitors (e.g., azathioprine, leflunomide, and mycophenolate), biologics (e.g., abatacept, adalimumab, anakinra, certolizumab, etanercept, golimumab, infliximab, ixekizumab, natalizumab, rituximab, secukinumab, tocilizumab, ustekinumab, and vedolizumab, and monoclonal antibodies (e.g., basiliximab and daclizumab).

This document also provides articles of manufacture (e.g., kits) containing one or more LUZP4 polypeptides described herein. LUZP4 polypeptides described herein that are included in an article of manufacture can be provided within a cell, in a solution in which they are soluble, or in a lyophilized form. The kit may further include a second substance that, for example, provides for detection of a LUZP4 polypeptide/anti-LUZP4 autoantibody complex. Such substances can be an anti-LUZP4 antibody, an anti-IgG antibody (e.g., an anti-human IgG antibody), or a combination thereof. In some cases, such substances can include a covalently linked detectable label (e.g., a fluorescent label). In addition, a kit can include directions for using the LUZP4 polypeptides and/or directions for practicing a method described herein (i.e., detecting LUZP4-specific autoantibodies in a biological sample).

The invention will be further described in the following examples, which do not limit the scope of the invention described in the claims.

### EXAMPLES

### Example 1—Identification of LUZP4 IgG as a Marker for TGCT and Paraneoplastic Neurological Syndrome

Studies were conducted to evaluate archived serum or CSF specimens from patients. The patients originally were referred for evaluation of paraneoplastic encephalitis associated with TGCTs or testicular microlithiasis. Testicular cancer cases without paraneoplastic autoimmunity, normal controls, and other cancer and paraneoplastic disease controls also were tested. Human leukocyte antigen (HLA) analysis of patient DNA was performed by tissue typing using a reverse SSOP/sequence-based method.

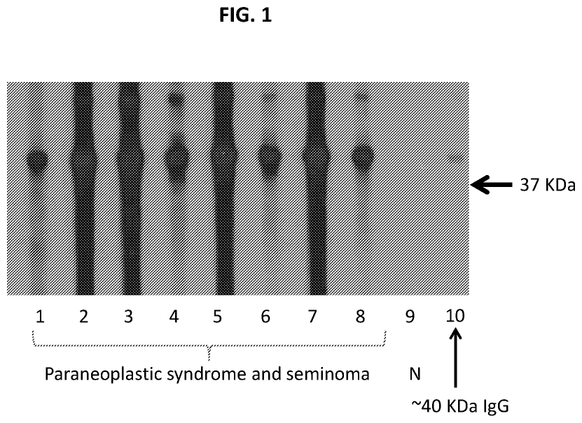

Immunoblot assays were performed with TCam-2 TGCT cell line antigen preparations to screen patient serum and CSF samples for a common autoantigen. A 37-40 KDa protein was identified in the majority of screened serum samples from patients with TGCT and paraneoplastic syndrome (FIG. 1). Patient IgGs that bound to the membrane at the 37-40 KDa molecular weight were acid-eluted and used for immunoprecipitation (FIG. 1). High pressure liquid chromatography electrospray tandem mass spectrometry was utilized for protein identification. LUZP4 was identified as a protein that was detected in patient samples but not in control samples.

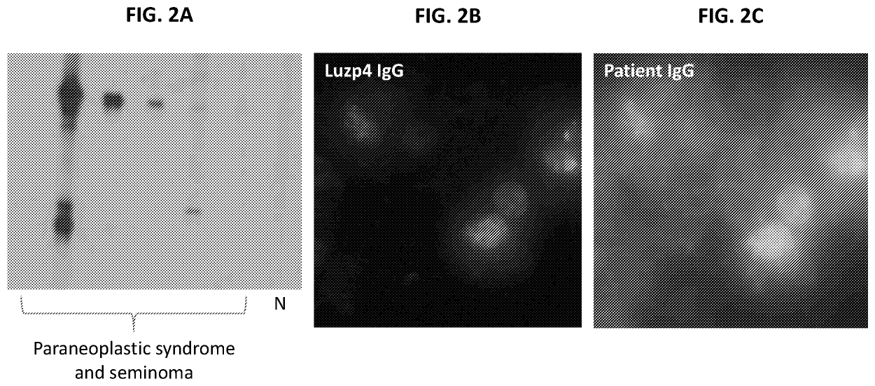

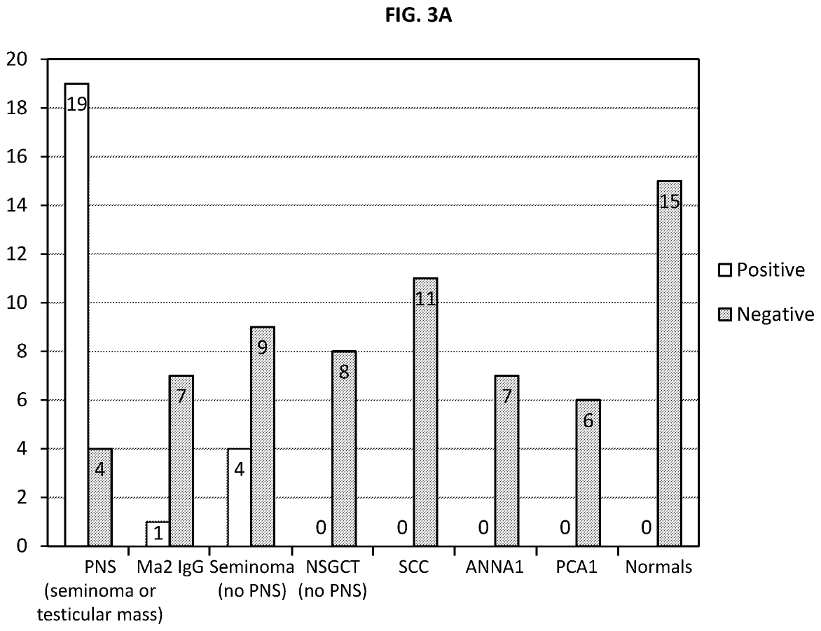

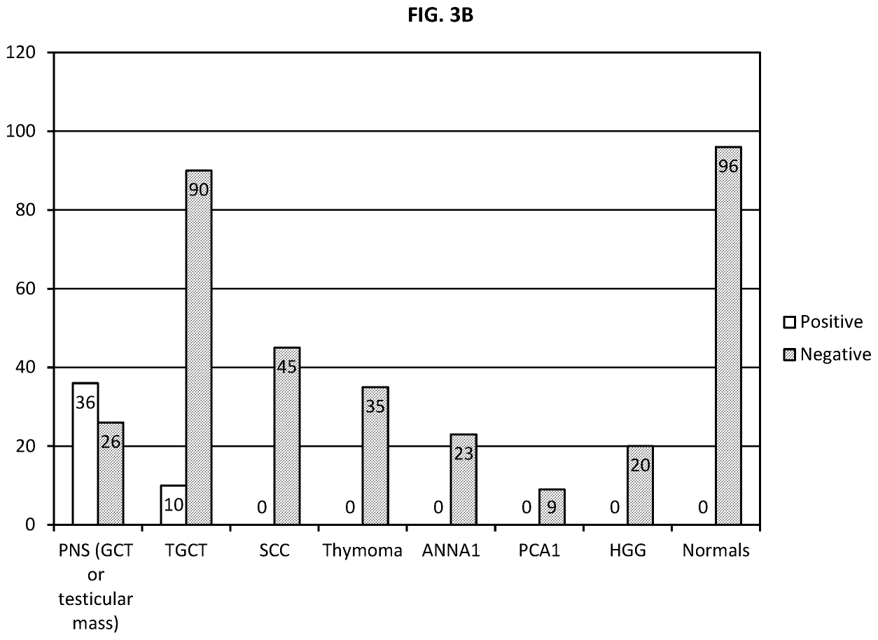

To determine whether LUZP4 was the specific autoantigen, a C-terminal DDK tag LUZP4 overexpression lysate was obtained, and patient and control samples were evaluated for autoantibody presence. Specificity was validated by immunoreactivity of patient IgG to a candidate protein overexpression lysate on western blot (FIG. 2A). The antibodies were further validated using a human embryonic kidney 293T cell LUZP4 overexpression system (FIGS. 2B and 2C). LUZP4 IgG was detected in 19 of the 23 patients with paraneoplastic neurological syndrome associated with TGCT (n=14 of 18; testicular, 11; extra-testicular, 3) and/or testicular mass/microlithiasis without a characterized autoantibody (n=5 of 5) (FIG. 3A).

The antibodies also were validated using a LUZP4 ELISA. Immulon 2HB ELISA plates were coated with 10 ng of protein per well. Recombinant LUZ4 (Abnova) was diluted into 0.01 M NaPO₄ pH 7.4 and incubated for one hour at 37° C. The plates were then washed three times using PBS with 0.05% Tween-20. Sera tested in duplicate were centrifuged at 10,000 g, diluted to 1/50 in PBS with 0.05% Tween-20 and 10% goat serum, and incubated at 37° C. for one hour. After the plates were washed three times, secondary antibody (alkaline phosphatase-labeled goat F(ab)′ anti human IgG Fc, diluted in PBS with 0.05% Tween-20 and 10% goat serum at 1/4000) was added, and the plates were incubated at 37° C. for one hour. The plates were subsequently washed three times, and enzyme substrate was added and incubated for one hour at 37° C. The colorimetric reaction was stopped by addition of 1M NaOH. Plates were read at absorbance 405 nm. Positives had an O.D. two times higher than the average normal sera.

Using the ELISA assay, LUZP4 IgG was detected in 26 of 36 patients with paraneoplastic neurological syndrome (PNS) associated with TGCT (n=22; testicular, 14; extra-testicular, 8) and/or testicular mass/microlithiasis without a characterized autoantibody (n=4) (FIG. 3B). In addition, two women with germ cell tumors (ovarian teratomas, 2 of 22 tested, 7%) were positive for LUZP4 IgG. The median age of symptom onset was 45 years (range: 24-84 years). The most common neurological phenotype among these cases was paraneoplastic rhomenhencephalitis (n=19, 68%). Other presentations included paraneoplastic limbic encephalitis (n=5, 18%) and paraneoplastic polyradiculoneuropathy (n=4, 14%). Some patients had multifocal neurological involvents during the disease course, such as rhombencephalitis and neuropathy (n=5, 18%), and limbic encephalitis and neuropathy (n=1, 4%). Ataxia was a common presenting symptom (n=17, 61%). Other common neurological signs or symptoms included nystagmus (n=11, 39%), diplopia (n=11, 39%), dysmetria (n=17, 46%), hearing loss (n=11, 39%), encephalopathy (n=10, 36%), seizures (n=6, 21%), and tremors (n=6, 21%). Nearly all patients with CSF evaluation had inflammatory CSF (22/23, 96%). A human leukocyte antigen (HLA) analysis was conducted for five seropositive patients with TGCT and paraneoplastic syndrome, revealing the presence of HLA DR17-DQ2 in four of the five (80%).

The majority of LUZP4 IgG patients also were positive for KLHL11 IgG (n=19, 68%). One LUZP4 IgG seropositive case also had Ma2 IgG detected (4%). LUZP4 IgG also was detected in the serum of 10 of 90 testicular germ cell tumor patients (11%, seminoma [n=8], embroyonal cancer [n=2]) without paraneoplastic neurological syndrome. Other paraneoplastic controls (ANNA1 IgG seropositive [n=23], PCA1 [n=9]), cancer controls (small cell cancer [n=45], thymoma [n=31]), hypergamma globulinemia (n=20) and normal controls (n=96) were all negative.

From these studies, it was concluded that LUZP4 IgG (also known as cancer testes antigen (CTA) 28 IgG) is a specific serological biomarker of TGCT and associated paraneoplastic autoimmunity. A minority of TGCT patients without paraneoplastic disorders also were positive for LUZP4 IgG. As a fetal embryonic antigen, LUZP4 may elicit specific T-cell responses that are useful for diagnosis and potential treatment. Immunological testing of LUZP4 IgG therefore may aid in early diagnosis and management of underlying TGCTs, especially metastatic or extra-testicular cases, subsequently impacting clinical outcomes. For example, in patients with imaging that indicates possible residual viable TGCT after chemotherapy in the retroperitoneal lymph nodes (most common), lungs, or mediastinum, the most challenging decision for the clinician is often whether the lesions harbor active TGCT, and thereby whether they require surgical resection (Mosharafa et al., supra). Use of the LUZP4 biomarker therefore can be very helpful in determining whether a patient is truly free of active TGCT or if the patient needs additional treatment, particularly surgery.

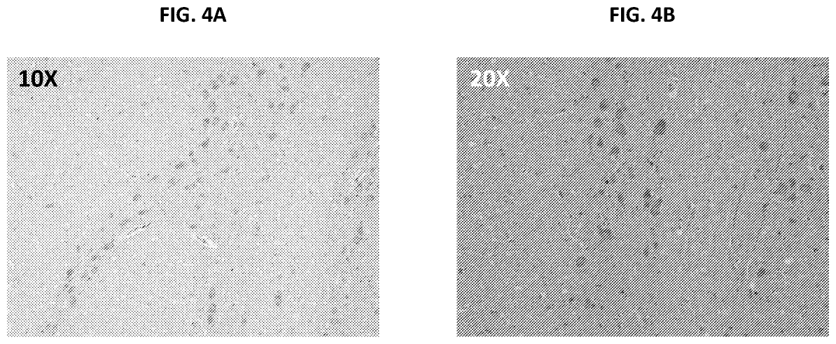

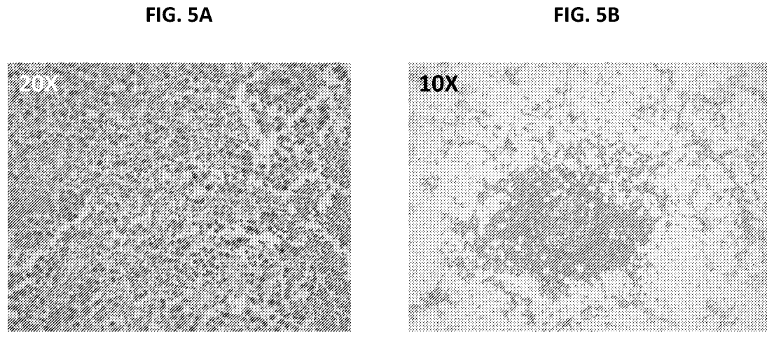

Expression of LUZP4 seems to be limited to testes and cells with intrinsic pluripotent properties, although immunohistochemistry studies of human brain demonstrated that it may have some expression in the brain as well (FIGS. 4A and 4B). LUZP4 was detected in a TGCT patient via serological analysis of cDNA expression libraries (Tureci et al., Oncogene, 21(24):3879-3888, 2002), but its utility as a serological biomarker of TGCT or paraneoplastic autoimmunity only became apparent when the studies described herein were conducted. The data presented herein highlight the specificity of LUZP4 IgG for TGCTs. Additionally, the rate of positivity is considerably higher in the setting of paraneoplastic autoimmunity, which is most likely propagated by anti-cancer immune response (FIGS. 5A and 5B).

### Example 2—Characteristics of LUZP4 IgG as a Marker for TGCT and Paraneoplastic Autoimmunity

To further evaluate the specific characteristics of LUZP4 IgG as a marker for TGCT and paraneoplastic autoimmunity, serum samples are collected from urology, oncology, and autoimmune neurology patients with testicular germ cell tumors, with or without paraneoplastic neurological syndrome. Inclusion criteria include testicular tumors (TGCT or mixed germ cell tumors) and patient age of at least 18 years. Serum samples are collected over a period of six months. For questions regarding testicular tumor histology and tumor infiltrating lymphocytes, tumor slides are reviewed by an anatomic pathologist.

Collected serum samples are tested by immunoblot for LUZP4 IgG serostatus. PBMC and DNA collection kits are provided to patients who are seropositive for LUZP4 IgG. PBMC samples are analyzed for LUZP4 specific T-cell responses by ELISpot assays and CyTOF. DNA samples are evaluated for common HLA associations by tissue typing using reverse SSOP or sequence based typing methods.

To evaluate the use of LUZP4 IgG as a marker of remission or recurrence in seropositive cases, post-treatment (chemotherapy, radiation, surgery, immunosuppressive therapy, or combinations thereof) samples are collected for LUZP4 IgG seropositive cases. Seroconversion to LUZP4 IgG negative status after treatment suggests the utility of LUZP4 IgG as a marker of remission, and subsequent conversion to LUZP4 IgG seropositive status suggests the use of LUZP4 IgG as a marker of recurrence.

The intracellular localization of LUZP4 suggests that the antibody itself is not pathogenic in terms of paraneoplastic autoimmunity. LUZP4 IgG may be a marker of a T-cell specific cytotoxic immune response, as the literature suggests antigen specific T-cell mediated “immune-surveillance” of tumors (see, e.g., Liu et al., Cancer Biother Radiopharm 20(5):491-501, 2005; and Albert et al., Nat Med 4(11):1321-1324, 1998). Studies are conducted to evaluate whether the presence of a LUZP4 specific cytotoxic T-cell lymphocyte response is correlated with the presence of tumor-infiltrating lymphocytes (FIG. 4B) and better malignancy outcomes. It also is possible that antigen specific T-cell activity is inversely related to the severity of paraneoplastic neurological outcome, and/or may be a useful avenue for cancer immune therapy without exacerbating the autoimmune pathology.

To reveal the therapeutic potential of targeting LUZP4 autoantigens for control of paraneoplastic disease, patient lymphocytes are evaluated for a LUZP4 specific T-cell response. Immunophenotyping experiments and functional assays such as CyTOF, ELISpot analysis, and cell-based T-cell inhibition assays are used to define the dual roles of autoreactive cytotoxic T-cells and regulatory T cells in autoimmune pathology and cancer immune surveillance in paraneoplastic disease. Information gleaned from these studies is used to investigate novel therapeutic strategies, including stimulation of specific regulatory T cells for management-refractory autoimmune neurological conditions. These studies also can provide novel insights into mechanisms that regulate cell mediated autoimmunity in neuroimmunological conditions and cancers in general.

In paraneoplastic neurological syndromes, auto-antibody titers typically have no correlation with disease severity (particularly antibodies that are produced against intracellular antigens). The post-immunotherapy LUZP4 specific T-cell population is evaluated in the subset of patients with paraneoplastic autoimmunity in order to delineate whether there is a relationship between the LUZP4 specific T-cell response and neurological outcomes following immunotherapy. Additionally, these studies can serve as a tool to predict relapse in patients with paraneoplastic syndromes.

Studies of paraneoplastic syndromes suggest that HLA associations are tumor specific (Hillary et al., J Neuroimmunol, 315:28-32, 2018). A common serological marker and strong cancer association with TGCT suggests that the LUZP4-associated immune response may be mediated by specific HLA subtypes. The data described herein show a strong association with HLA DR17-DQ2. A larger data set is analyzed to confirm this finding; the finding also is compared with genome-wide association studies of testicular germ cell tumors (Rapley et al., Nat Genet, 41(7):807-810, 2009) to determine whether the finding is secondary to the common immune response or secondary to HLA association in the cancer.

In addition, clinically useful tests are developed and validated. These tests are utilized in urology and oncology clinics as part of the work up for patients presenting with a scrotal mass, or in the setting of untreated or treated metastatic TCGT. Further tests are developed and validated for inclusion in a panel of paraneoplastic antibodies offered by neuroimmunology laboratories.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of detecting the presence or absence of a LUZP4-specific autoantibody in a biological sample from a mammal, wherein said method comprises:
(a) contacting said biological sample with a LUZP4 polypeptide to form a LUZP4/LUZP4-specific autoantibody complex if said biological sample contains LUZP4-specific autoantibodies; and
(b) detecting the presence or absence of said complex.

2. The method of claim 1, wherein said method comprises detecting the presence of said complex.

3. The method of claim 2, wherein the presence of said LUZP4-specific autoantibody in said biological sample is associated with germ cell tumor or germ cell tumor-associated paraneoplastic neurological syndrome associated with LUZP4-specific autoantibodies in said mammal.

4. The method of claim 3, wherein said paraneoplastic neurological syndrome is a paraneoplastic encephalitis.

5. The method of claim 1, wherein said method comprises performing a Western blot to detect said complex.

6. The method of claim 1, wherein said biological sample is selected from the group consisting of whole blood, serum, plasma, peripheral blood mononuclear cells (PBMCs) and cerebrospinal fluid.

7. A kit comprising a LUZP4 polypeptide and an anti-IgG antibody.

8. The kit of claim 7, wherein said anti-IgG antibody is an anti-human IgG antibody.

9. The kit of claim 8, wherein said anti-human IgG antibody comprises a covalently attached label.

10. The kit of claim 7, wherein said kit comprises an anti-LUZP4 antibody.

11. A method of treating a mammal having a germ cell tumor or germ cell tumor associated paraneoplastic autoimmunity associated with LUZP4-specific autoantibodies, wherein said method comprises:
(a) withdrawing a biological fluid from the mammal, wherein the biological fluid contains LUZP4-specific autoantibodies;
(b) contacting said biological fluid with a LUZP4 polypeptide to remove a substantial portion of said LUZP4-specific autoantibodies from said biological fluid; and
(c) returning the biological fluid to the mammal.

12. The method of claim 11, wherein said mammal is a human.

13. The method of claim 11, wherein said biological fluid comprises blood.

14. The method of claim 11, wherein said mammal has germ cell tumor or germ cell tumor-associated paraneoplastic autoimmunity.

15. The method of claim 14, wherein said germ cell tumor or germ cell tumor-associated paraneoplastic autoimmunity is paraneoplastic encephalitis.

16-19. (canceled)


SEQUENCE LISTING (4 sequences)

SEQ ID NO: 1 - protein, 313 aa, Homo sapiens
MASFRKLTLSEKVPPNHPSRKKVNFLDMSLDDIIIYKELEGTNAEEEKNKRQNHSKKESPSRQQSKAHRHRHRRGYSRCRSNSEEGNHDKKPSQKPSGFKSGQHPLNGQPLIEQEKCSDNYEAQAEKNQGQSEGNQHQSEGNPDKSEESQGQPEENHHSERSRNHLERSLSQSDRSQGQLKRHHPQYERSHGQYKRSHGQSERSHGHSERSHGHSERSHGHSERSHGHSKRSRSQGDLVDTQSDLIATQRDLIATQKDLIATQRDLIATQRDLIVTQRDLVATERDLINQSGRSHGQSERHQRYSTGKNTITT

SEQ ID NO: 2 - protein, 231 aa, Homo sapiens
MLKKKRIKDRTIVKRNRLQDSNQKLIDIAIGEEKCSDNYEAQAEKNQGQSEGNQHQSEGNPDKSEESQGQPEENHHSERSRNHLERSLSQSDRSQGQLKRHHPQYERSHGQYKRSHGQSERSHGHSERSHGHSERSHGHSERSHGHSKRSRSQGDLVDTQSDLIATQRDLIATQKDLIATQRDLIATQRDLIVTQRDLVATERDLINQSGRSHGQSERHQRYSTGKNTITT

SEQ ID NO: 3 - DNA, 1746 bp, Homo sapiens
acacgcgccctacctcggagtgtgtggcgccatgatgcagggaagatggcttcgtttcggaagctaacgctttctgaaaaagtgccgccaaatcatcccagtcggaaaaaggttaacttcctagatatgtctctagacgacattataatctataaagagttagaagggacaaatgctgaagaagaaaagaataaaagacagaaccatagtaaaaaggaatcgccttcaagacagcaatcaaaagctcatagacatcgccatcggagaggctactcaagatgcagaagcaactctgaggaaggaaatcatgataaaaaaccatcccaaaaaccttctggattcaagtctggacaacaccctttaaatgggcagcctttaattgagcaggagaagtgcagtgacaattatgaggcccaagcagagaagaatcaaggccagtcagaggggaaccagcatcaatcagaaggaaatccggacaaatcagaagaatcccagggccaaccagaagaaaatcatcattctgagcgatcccgaaaccacttagagagatctctttctcagtcagacagatctcaagggcagctaaagagacatcatccccaatatgagagatctcatggccaatacaagagatctcatggtcaatctgagagatctcatggccactcagagagatctcatggtcactcagagagatctcatggtcactcagagagatctcatggtcactcaaagagatctcgtagccagggagatcttgtggacactcagagtgatctcatagccactcagagagatctcatagccactcagaaagatctcatagccactcagagagatctcatagccactcagagagatctcatagtcactcagagagatctcgtggccactgagagagatctcataaatcagtcagggagatctcatggccaatcagaaagacatcagagatactcaacaggtaaaaatacaataactacttaatcatcagaacaatgtgttgaattctgtggaaatagaaaagcatatatctatattctaatggctaaatatgtatttgttgaaacatgtatattgggacaaagacataaatattagaatggaggtaatacatacatagtatcaatattgtttcaacttgatgtcctctaagctatcatccagttacccaggatgtcccattaagttgttcccggtaggtctgctttccctggaagagccgtatgtactcagcctttcctattgggccttccccacaattagaatattttgacttagtgtcctgtcccccttggacgttccaacttgacttagtgtccagtgccccttggacattccaacctggtaggtaagctaatctaacaactaactgccaaattgataatatataatctatgataatgaatatctcttttgtgtctccttcctaagccatcctcagagagtccttagcagacaaatggtagatgtatctttgggcagctgaacttttctgctttcctcaaatcagaccatatgagaggatatattctatgcatagatgtaatgctaaccttctgaatatattttgaatacatttatatattcactgttgccttataaaactgttagggtaggtctgtctaccctagcaaaagaaacacagaaatttaaatgtactgggagttatgttgttaaaaacacaagatatgttaactgcagtttgtttggttattcaataaaagttttagttttaatttctca

SEQ ID NO: 4 - DNA, 1635 bp, Homo sapiens
acgcgccctacctcggagtgtgtggcgccatgatgcagggaagatggcttcgtttcggaagctaacgctttctgaaaaagtgccgccaaatcatcccagtcggaaaaaggttaacttcctagatatgtctctagacgacattataatctataaagagttagaagggacaaatgctgaagaagaaaagaataaaagacagaaccatagtaaaaaggaatcgccttcaagacagcaatcaaaagctcatagacatcgccatcggagaggagaagtgcagtgacaattatgaggcccaagcagagaagaatcaaggccagtcagaggggaaccagcatcaatcagaaggaaatccggacaaatcagaagaatcccagggccaaccagaagaaaatcatcattctgagcgatcccgaaaccacttagagagatctctttctcagtcagacagatctcaagggcagctaaagagacatcatccccaatatgagagatctcatggccaatacaagagatctcatggtcaatctgagagatctcatggccactcagagagatctcatggtcactcagagagatctcatggtcactcagagagatctcatggtcactcaaagagatctcgtagccagggagatcttgtggacactcagagtgatctcatagccactcagagagatctcatagccactcagaaagatctcatagccactcagagagatctcatagccactcagagagatctcatagtcactcagagagatctcgtggccactgagagagatctcataaatcagtcagggagatctcatggccaatcagaaagacatcagagatactcaacaggtaaaaatacaataactacttaatcatcagaacaatgtgttgaattctgtggaaatagaaaagcatatatctatattctaatggctaaatatgtatttgttgaaacatgtatattgggacaaagacataaatattagaatggaggtaatacatacatagtatcaatattgtttcaacttgatgtcctctaagctatcatccagttacccaggatgtcccattaagttgttcccggtaggtctgctttccctggaagagccgtatgtactcagcctttcctattgggccttccccacaattagaatattttgacttagtgtcctgtcccccttggacgttccaacttgacttagtgtccagtgccccttggacattccaacctggtaggtaagctaatctaacaactaactgccaaattgataatatataatctatgataatgaatatctcttttgtgtctccttcctaagccatcctcagagagtccttagcagacaaatggtagatgtatctttgggcagctgaacttttctgctttcctcaaatcagaccatatgagaggatatattctatgcatagatgtaatgctaaccttctgaatatattttgaatacatttatatattcactgttgccttataaaactgttagggtaggtctgtctaccctagcaaaagaaacacagaaatttaaatgtactgggagttatgttgttaaaaacacaagatatgttaactgcagtttgtttggttattcaataaaagttttagttttaaaaaaaaaaaaaaaaaaa